(19) United States
(12) Patent Application Publication
(10) Pub. No.: US 2007/0293717 A1
(43) Pub. Date: Dec. 20, 2007

(54) TUBE MESH FOR ABDOMINAL SACRAL COLPOPEXY

(75) Inventors: Richard C. Kaleta, Plymouth, MN (US); John Jason Buysman, Minnetonka, MN (US); Kelly Ann Dockendorf, St. Paul, MN (US); Matthew J. Olson, Crystal, MN (US); James E. Cox, Corcoran, MN (US)

Correspondence Address: OBLON, SPIVAK, MCCLELLAND MAIER & NEUSTADT, P.C., 1940 DUKE STREET, ALEXANDRIA, VA, 22314, US

(73) Assignee: AMS Research Corporation, Minnetonka, MN (US)

(21) Appl. No.: 11/748,197

(22) Filed: May 14, 2007

Related U.S. Application Data
(60) Provisional application No. 60/799,675, filed on May 12, 2006.

(51) Int. Cl. A61F 2/00

(52) U.S. Cl. 600/37

(57) ABSTRACT

Improved methods and apparatuses for treatment of pelvic organ prolapse are provided. A specialized sacral colpopexy mesh having a mesh cylinder attached to a first end of a main mesh is disclosed, and a method for use thereof in abdominal sacral colpopexy. A novel connector that is used to attach a mesh to the needle, including gripping features that improve the grip and allowing for easier connection and disconnection is disclosed, as well as a novel method and apparatus for connecting a mesh to a needle.

### CROSS REFERENCE TO RELATED APPLICATIONS

The present application claims priority to U.S. Provisional Application No. 60/799,675, filed May 12, 2006, the entire contents of which are herein incorporated by reference.

### BACKGROUND OF THE INVENTION

1. Field of the Invention

This invention relates to urogenital surgery.

2. Description of the Related Art

Female genital prolapse has long plagued women. It is estimated by the U.S. National Center for Health Statistics that 247,000 operations for genital prolapse were performed in 1998. With the increasing age of the U.S. population, these problems will likely assume additional importance.

Vaginal prolapse develops when intra-abdominal pressure pushes the vagina outside the body. In a normal situation, the levator ani muscles close the pelvic floor. This results in little force being applied to the fascia and ligaments that support the genital organs. Increases in abdominal pressure, failure of the muscles to keep the pelvic floor closed, and damage to the ligaments and fascia all contribute to the development of prolapse. In addition, if a woman has a hysterectomy, the vaginal angle may be altered, causing increased pressure at a more acute angle, accelerating the prolapse.

There are generally two different types of tissue that make up the supportive structure of the vagina and uterus. First, there are fibrous connective tissues that attach these organs to the pelvic walls (cardinal and uterosacral ligaments; pubocervical and rectovaginal fascia). Second, the levator ani muscles close the pelvic floor so the organs can rest on the muscular shelf thereby provided. It is when damage to the muscles open the pelvic floor or during the trauma of childbirth that the fascia and ligaments are strained. Breaks in the fascia allow the wall of the vagina or cervix to prolapse downward.

Several factors have been implicated as being involved in genital prolapse in women. It is thought that individual women have differing inherent strength of the relevant connective tissue. Further, loss of connective tissue strength might be associated with damage at childbirth, deterioration with age, poor collagen repair mechanisms, and poor nutrition. Loss of muscle strength might be associated with neuromuscular damage during childbirth, neural damage from chronic straining, and metabolic diseases that affect muscle function. Other factors involved in prolapse include increased loads on the supportive system, as seen in prolonged lifting or chronic coughing from chronic pulmonary disease, or some disturbance in the balance of the structural support of the genital organs. Obesity, constipation, and a history of hysterectomy have also been implicated as possible factors.

The common clinical symptoms of vaginal prolapse are related to the fact that, following hysterectomy, the vagina is inappropriately serving the role of a structural layer between intra-abdominal pressure and atmospheric pressure. This pressure differential puts tension on the supporting structures of the vagina, causing a “dragging feeling” where the tissues connect to the pelvic wall or a sacral backache due to traction on the uterosacral ligaments. Exposure of the moist vaginal walls leads to a feeling of perineal wetness and can lead to ulceration of the exposed vaginal wall. Vaginal prolapse may also result in loss of urethral support due to displacement of the normal structural relationship, resulting in stress urinary incontinence. Certain disruptions of the normal structural relationships can result in urinary retention, as well. Stretching of the bladder base is associated with vaginal prolapse and can result in complaints of increased urinary urgency and frequency. Other symptoms, such as anal incontinence and related bowel symptoms, and sexual dysfunction are also frequently seen with vaginal prolapse.

Anterior vaginal wall prolapse causes the vaginal wall to fail to hold the bladder in place. This condition, in which the bladder sags or drops into the vagina, is termed a cystocele. There are two types of cystocele caused by anterior vaginal wall prolapse. Paravaginal defect is caused by weakness in the lateral supports (pubourethral ligaments and attachment of the bladder to the endopelvic fascia); central defect is caused by weakness in the central supports. There may also be a transverse defect, causing cystecele across the vagina.

Posterior vaginal wall prolapse results in descent of the rectum into the vagina, often termed a rectocele, or the presence of small intestine in a hernia sac between the rectum and vagina, called an enterocele. Broadly, there are four types based on suspected etiology. Congenital enteroceles are thought to occur because of failure of fusion or reopening of the fused peritoneal leaves down to the perineal body. Posthysterectomy vault prolapses may be “pulsion” types that are caused by pushing with increased intra-abdominal pressure. They may occur because of failure to reapproximate the superior aspects of the pubocervical fascia and the rectovaginal fascia at the time of surgery. Enteroceles that are associated with cystocele and rectocele may be from “traction” or pulling down of the vaginal vault by the prolapsing organs. Finally, iatrogenic prolapses may occur after a surgical procedure that changes the vaginal axis, such as certain surgical procedures for treatment of incontinence. With regard to rectoceles, low rectoceles may result from disruption of connective tissue supports in the distal posterior vaginal wall, perineal membrane, and perineal body. Mid-vaginal and high rectoceles may result from loss of lateral supports or defects in the rectovaginal septum. High rectoceles may result from loss of apical vaginal supports. Posterior or posthysterectomy enteroceles may accompany rectoceles.

As noted, vaginal prolapse and the concomitant anterior cystocele can lead to discomfort, urinary incontinence, and incomplete emptying of the bladder. Posterior vaginal prolapse may additionally cause defecatory problems, such as tenesmus and constipation.

Many techniques have been tried to correct or ameliorate the prolapse and its symptoms, with varying degrees of success. Nonsurgical treatment of prolapse involves measures to improve the factors associated with prolapse, including treating chronic cough, obesity, and constipation. Other nonsurgical treatments may include pelvic muscles exercises or supplementation with estrogen. These therapies may alleviate symptoms and prevent worsening, but the actual hernia will remain. Vaginal pessaries are the primary type of nonsurgical treatment, but there can be complications due to vaginal wall ulceration.

There are a variety of known surgical techniques for the treatment of anterior vaginal prolapses. In the small proportion of cases in which the prolapse is caused by a central defect, anterior colporrapphy is an option. This surgery involves a transvaginal approach in which plication sutures are used to reapproximate the attenuated tissue across the midline of the vagina. More commonly, the prolapse is due to a lateral defect or a combination of lateral and central defects. In these instances, several surgical techniques have been used, such as a combination of an anterior colporrapphy and a site-specific paravaginal repair. Both abdominal and vaginal approaches are utilized. Biological or synthetic grafts have been incorporated to augment repair.

Likewise, the treatment of posterior vaginal prolapses may vary. If symptoms are minimal, nonoperative therapy such as changes in activities, treatment of constipation, and Kegel exercises might be appropriate. Again, both vaginal and abdominal approaches are used, involving sutures to reapproximate the attenuated tissue and possibly a biological or synthetic graft to augment the repair.

Sacral colpopexy entails attaching the vaginal vault to the sacrum by use of mesh or fascia. The surgery may be performed through an abdominal incision or laparoscopically. Complications include mesh infection, mesh erosion, bowel obstruction, and hemorrhage from the presacral venous complex. If synthetic mesh is used, it is typically carefully customized or assembled into a special shape by the surgeon.

The abdominal sacral colpopexy is one of the most successful operations for vaginal vault prolapse with excellent results. The procedure of sacral colpopexy is complex in its nature and requires great expertise for a favorable outcome. Sacral colpopexy can be a tedious, challenging surgical procedure, with an average procedure length of 247 minutes reported in Winters et al, Abdominal Sacral Colpopexy and Abdominal Enterocele Repair in the Management of Vaginal Vault Prolapse, Urology 56 (Suppl 6A) (2000): 55-63. Some of this time is attributed to the time required for the surgeon to fashion the implant. In addition, it is often required to correct multiple pelvic floor abnormalities simultaneously, further increasing surgical time. Longer surgical times are often associated with increased complications, and increased costs, both in terms of morbidity and economics. Clearly, there is a need for modifications of instrumentation and technique to facility shortened procedure duration.

### SUMMARY OF THE INVENTION

The present invention includes surgical instruments and implantable articles for urological applications, particularly abdominal sacral colpopexy for the repair of pelvic floor defects.

In the usual sacral colpopexy procedure, polypropylene mesh is fashioned into a Y-shape, creating an anterior and posterior leaf. The mesh is then introduced into the abdomen. The anterior leaf of the mesh is sutured to the pubocervical fascia. The posterior leaf is then sutured to the rectovaginal fascia. The free end of the mesh is then attached to the anterior longitudinal ligament of the sacrum. The present invention comprises, in one aspect, a specialized sacral colpopexy mesh. The sacral colpopexy mesh comprises a mesh typical of the mesh implants used in sacral colpopexy. The sacral colpopexy mesh further comprises a mesh cylinder attached to a first end of the mesh. The mesh cylinder is constructed of compliant mesh material. The mesh cylinder can be placed over the vaginal apex intra-operatively, and attached with fewer sutures than the typical Y-shaped mesh.

Another aspect of the present invention is specially adapted instrumentation to facilitate alternative locations for the sacral ligament attachment for support in the usual sacral colpopexy. A mesh with a sheath may be used in such an alternative method. An aspect of the present invention is a novel method for connecting the mesh with the sheath to a needle. The mesh/needle attachment in this aspect of the present invention must be strong enough so that it does not detach during implantation of the mesh, but the attachment must be sufficiently detachable to allow for relative ease in removing once the needle has to be retracted. In this aspect, the mesh and sheath are overmolded by a polypropylene tip. The needle is slipped into the sheath and inserted into the polypropylene tip.

Another aspect of the present invention is a novel connector that is used to attach the mesh to the needle. The connector is adapted to be manually connected onto and removed from the needle by clockwise or counterclockwise rotation about the axis of the connector and needle. The small diameter of the connector makes this manual procedure somewhat difficult. In this aspect of the invention, the connector comprises gripping features that improve the grip, allowing for rotation with greater ease. These gripping features might comprise ridges or other textures. These textures also have the advantage of providing for tactile feedback that will assist the operator in determining the correct tightness of connection, i.e., prevent over-tight or lose connections.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 shows a step in the prior art method of sacral colpopexy, in which the anterior leaf of a mesh is attached to the pubocervical fascia.

FIG. 2 shows another step in the prior art method of sacral colpopexy, in which the posterior leaf being sutured to the rectovaginal fascia.

FIG. 3 shows another step in the prior art method of sacral colpopexy, in which the free end of the mesh is attached to the anterior longitudinal ligament of the sacrum.

FIG. 4 shows a view of the adapted needle tip of the present invention.

FIG. 5 shows a view of the over-molded polypropylene tip and the needle of the present invention.

FIG. 6 shows a view of the needle engaged with the plastic sheath of the present invention.

FIG. 7 shows a plan view of the present invention.

FIG. 8 shows another aspect of the present invention.

FIG. 9 shows the novel connector of the present invention.

FIG. 10 shows another view of the novel connector of the present invention.

FIG. 11 shows a view of the novel gripping feature of the present invention.

FIG. 12 shows another embodiment of the novel gripping feature of the present invention.

### DETAILED DESCRIPTION OF THE PREFERRED EMBODIMENTS

Referring now to the drawings, wherein like reference numerals designate identical or corresponding parts throughout the several views. The following description is meant to be illustrative only, and not limiting other embodiments of this invention will be apparent to those of ordinary skill in the art in view of this description.

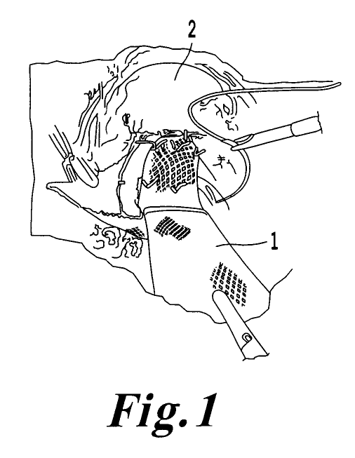

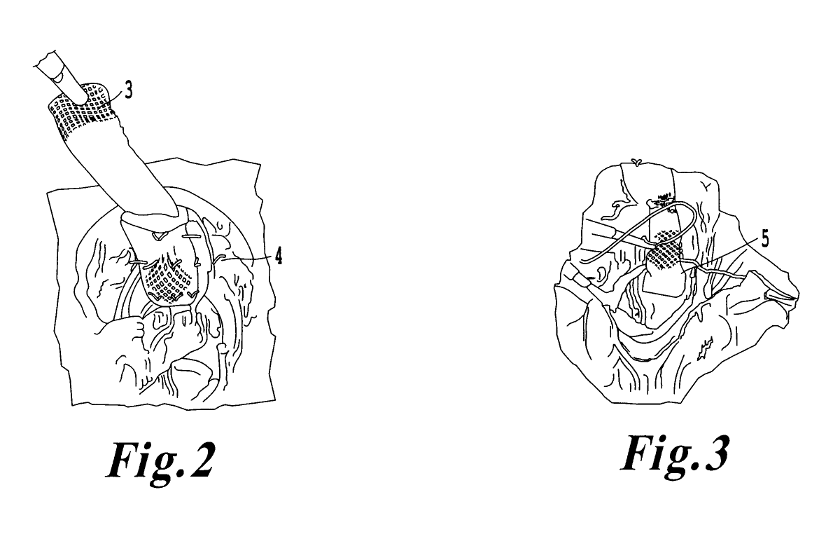

The sacral colpopexy procedure as customarily practiced is illustrated by FIGS. 1-3. As can be seen in FIG. 1, the anterior leaf of the y-shaped polypropylene mesh 1 is sutured to the pubocervical fascia 2. FIG. 2 shows the posterior leaf 3 being sutured to the rectovaginal fascia 4. The free end of the mesh is then attached to the anterior longitudinal ligament of the sacrum 5, as seen in FIG. 3. The present invention comprises, in one aspect, a specialized sacral colpopexy mesh. The sacral colpopexy mesh comprises a mesh typical of the mesh implants used in sacral colpopexy. The sacral colpopexy mesh further comprises a mesh cylinder attached to a first end of the mesh. The mesh cylinder is constructed of compliant mesh material. The mesh cylinder can be placed over the vaginal apex intra-operatively, and attached with fewer sutures than the typical Y-shaped mesh.

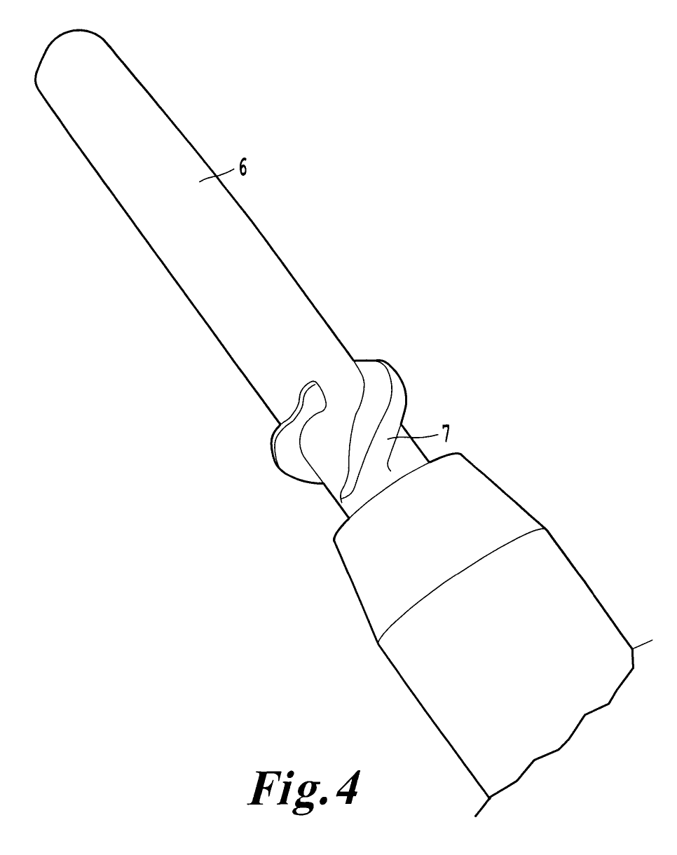

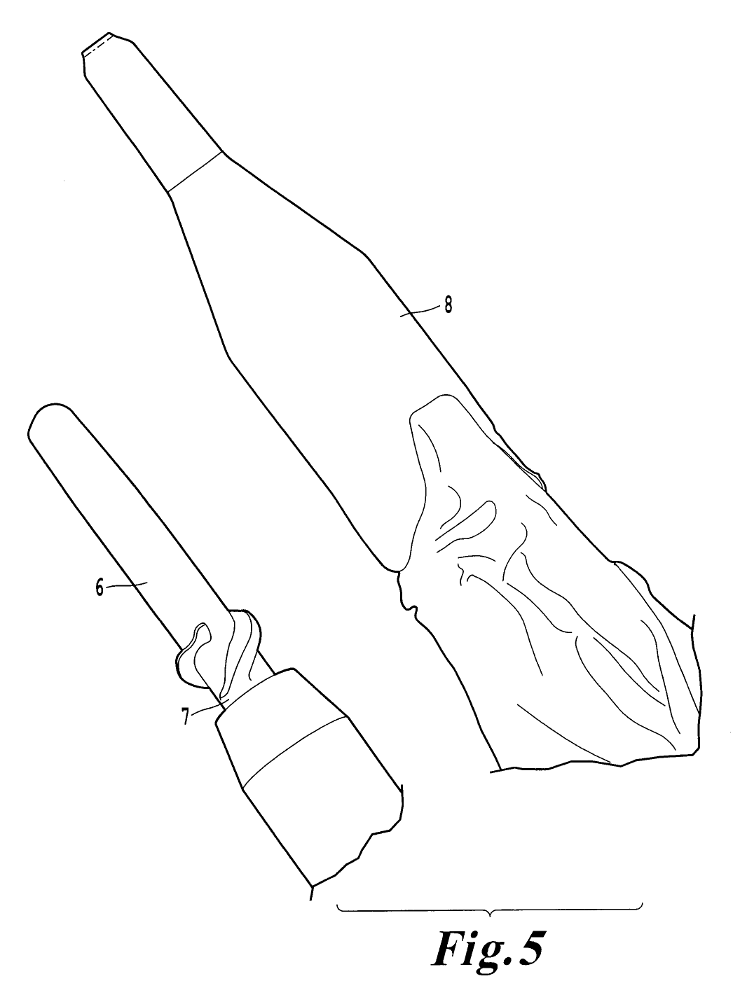

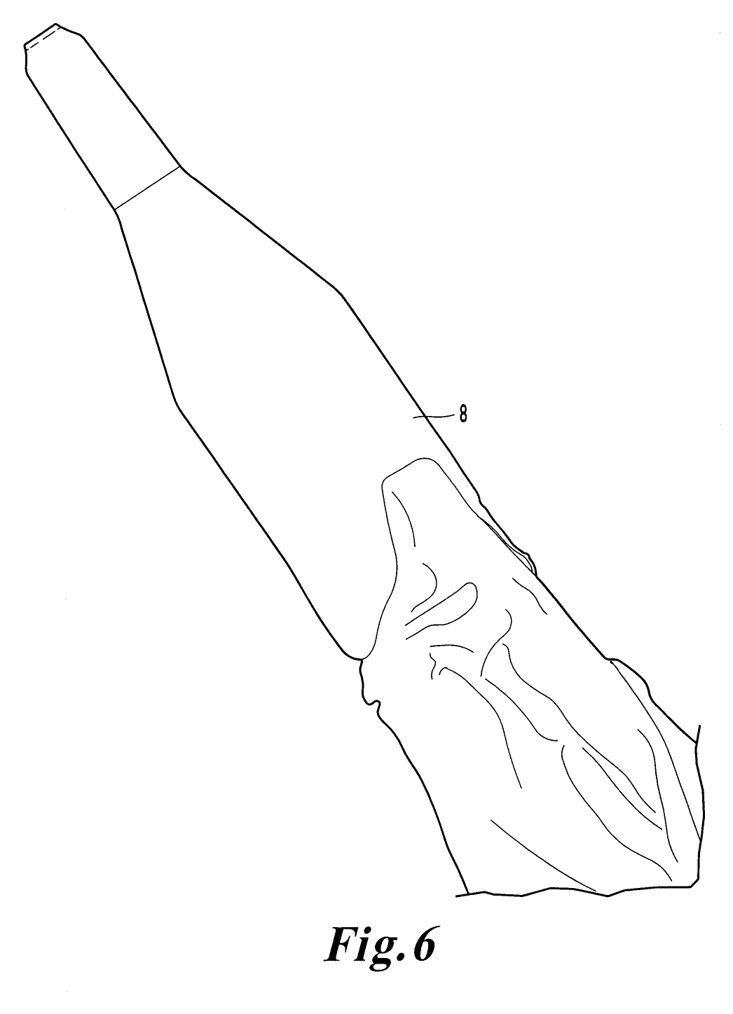

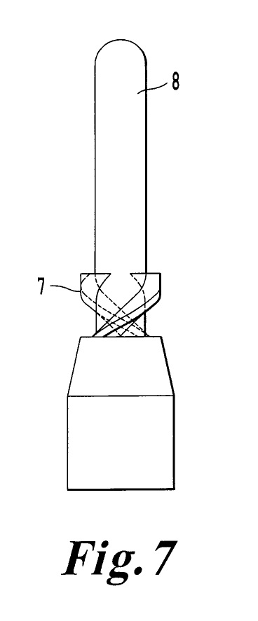

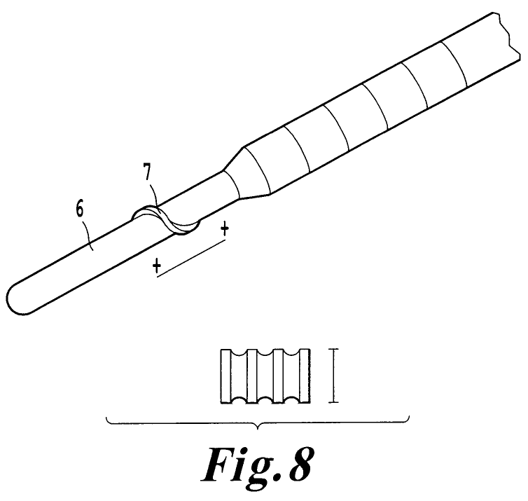

The novel instrumentation for connecting a mesh with a sheath to the needle of the present invention is shown in FIGS. 4-8. In an embodiment, the needle tip 6 has a six thread per inch helix 7 that runs about 0.075 inches in length, shown in FIGS. 4, 7, and 8. In operation, the needle is inserted about 0.275 inches into the over-molded non-helical polypropylene tip 8, shown in FIGS. 5 and 7, then twisted with a half turn to drive the about 0.095 inch major diameter of the helix into the polypropylene tip 8, as illustrated in FIGS. 6 and 7. With such a connection, the polypropylene tip is then secure enough to stay attached to the needle during implant, but easy enough to remove with a simple 180° counterclockwise twist.

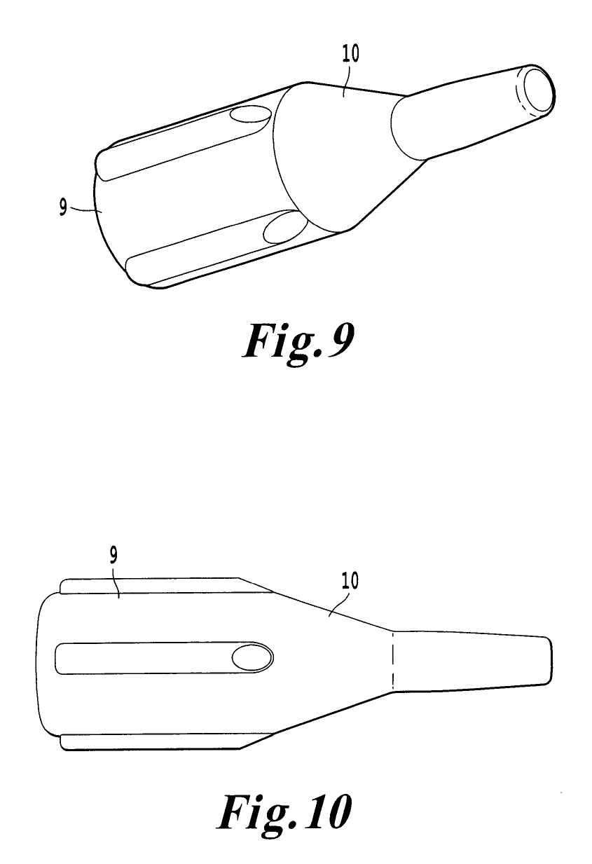

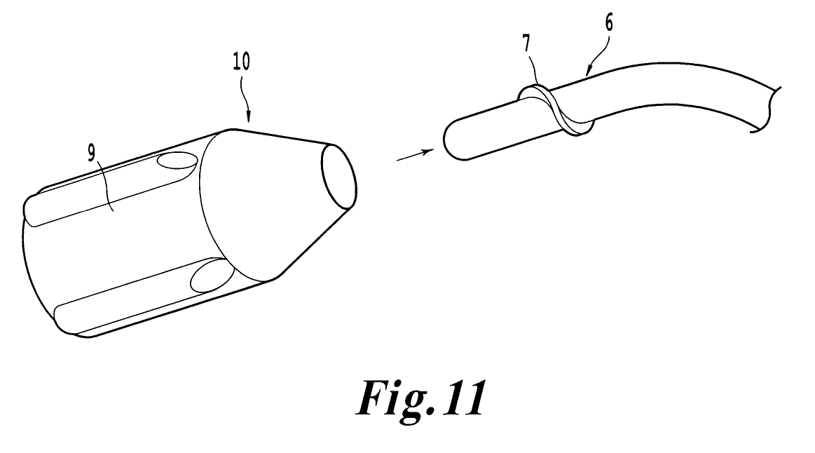

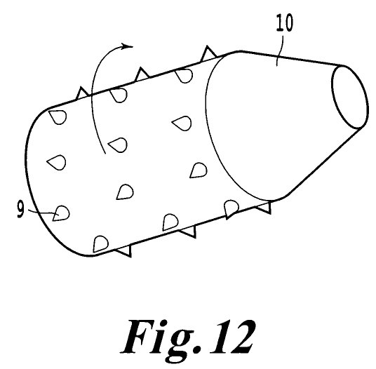

Another aspect of the present invention is a novel connector that is used to attach the mesh to the needle, shown in FIGS. 9 and 10. The connector 10 comprises gripping features 9 that improve the grip, allowing for rotation with greater ease. These gripping features 9 may comprise ridges, as illustrated in FIG. 11, or other textures, as illustrated in FIG. 12, or other textures useful for improving grip-ability of surgical instruments or other tools. The textures provide a convenient gripping surface and provide tactile feedback that will assist the operator in determining the correct tightness of the connection.

Obviously, numerous modifications and variations of the present invention are possible in light of the above teachings. It is therefore to be understood that within the scope of the appended claims, the invention may be practiced otherwise than as specifically described herein.

1. An apparatus for treating pelvic organ prolapse in a patient comprising:
a. a main mesh portion having first and second ends, and
b. a mesh cylinder attached to a first end of said main mesh portion.

2. The apparatus of claim 1, wherein said main portion is a mesh implant.

3. The apparatus of claim 1, wherein said mesh cylinder is adapted to be placed over the vaginal apex intra-operatively.

4. A connector adapted to detachably connect a mesh to a needle.

5. The connector of claim 4 wherein said connector is of an oblong cylindrical shape and has first and second ends.

6. The connector of claim 4 comprising at least one gripping feature.

7. The connector of claim 6, wherein said gripping feature comprises ridges,.

8. The connector of claim 6, wherein said gripping feature comprises raised bumps.

9. A combination comprising the apparatus of claim 1 and a connector adapted to connect the apparatus to a needle.

10. The combination of claim 9, wherein said connector comprises a sheath associated with said apparatus.

11. The combination of claim 10, wherein said sheath is formed by overmolding.

12. The combination of claim 10, wherein said sheath comprises polypropylene.

13. The combination of claim 9, wherein said connector is of an oblong cylindrical shape and has first and second ends.
